(19) United States
(12) Patent Application Publication
(10) Pub. No.: US 2026/0212988 A1
(43) Pub. Date: Jul. 23, 2026

(54) PARAMETER CHECK SYSTEM, TREATMENT PLAN VERIFICATION SYSTEM, TREATMENT PLANNING SYSTEM, RADIOTHERAPY SYSTEM, PROGRAM PRODUCT, AND PARAMETER CHECK METHOD

(71) Applicant: HITACHI HIGH-TECH CORPORATION, Tokyo (JP)

(72) Inventors: Mayu OSUGI, Tokyo (JP); Takahiro YAMADA, Tokyo (JP); Rintarou FUJIMOTO, Tokyo (JP)

(21) Appl. No.: 19/157,869

(22) PCT Filed: Jul. 5, 2023
(86) PCT No.: PCT/JP2023/025042

§ 371 Date: Aug. 19, 2025

(30) Foreign Application Priority Data
Mar. 23, 2023 (JP) 2023-046532

(51) Int. Cl. G16H 20/40; A61N 5/10

(52) U.S. Cl. CPC G16H 20/40; A61N 5/1039; A61N 5/1045; A61N 5/1048; A61N 2005/1074

(57) ABSTRACT

Provided are: a storage unit 341 structured to identify and to store an immutable parameter that should not change and a mutable parameter that may change, between parameters of a first treatment plan and parameters of a second treatment plan; and a comparison unit 342 structured to compare, among the plurality of parameters stored in the storage unit 341, at least either an event regarding whether or not the immutable parameter matches between the first treatment plan and the second treatment plan, or an event regarding whether or not variation of the mutable parameter falls within a prescribed range. This provides a parameter check system, a treatment plan verification system, a treatment planning system and a radiotherapy system, a program product, and a parameter check method, all capable of reducing burdens on operators and patients in radiotherapy.

### TECHNICAL FIELD

The present invention relates to a parameter check system, a treatment plan verification system, a treatment planning system, a radiotherapy system, a program product, and a parameter check method.

### BACKGROUND ART

NPL 1 describes a technique for verifying that a large amount of electronic data, in the entire process of radiotherapy including particularly error-prone treatment planning processes, is genuine as intended by a radiation oncologist, and for practically ensuring and allowing the data to drive a radiotherapy system as intended, and to correctly accomplish dosage to patient.

### CITATION LIST

### Non-Patent Literature

NPL 1: Takeo Nakashima, 1. Considerations for Radiation Therapy Planning from Plan/Chart Check, Japanese Journal of Radiological Technology, 2022, Vol. 78, No. 4, p. 401-411

### SUMMARY OF INVENTION

### Technical Problem

In radiotherapy, prescription and treatment policy are determined for each patient, a treatment plan is then created, according to which radiation is irradiated over a plurality of days.

Note now, due to time elapsed from the date of treatment planning and the actual date of irradiation, an internal structure of the body would change.

Among radiotherapies, adaptive treatment (ART: Adaptive RadioTherapy or Adaptive Radiation Therapy), designed to optimize treatment planning parameters in accordance with an acquired patient image, can adapt the treatment plan to daily change of the patient, thus enabling a treatment with less burden on the patient. Modification or re-creation of the treatment plan may occasionally be referred to as re-planning, hereinafter.

Among the adaptive treatment, off-line adaptive treatment (offline-ART) is designed to implement re-acquisition of patient image, re-planning, and verification of the re-planning, within intervals of a plurality of scheduled dates of irradiation. Online adaptive therapy (online-ART) is designed to acquire an image of the patient laid on a couch on the day of treatment, whether or not the re-planning is necessary is then determined, followed by re-planning and verification of the plan.

Regarding re-planning, NPL 1 describes efficient use of a manual check list for ensuring consistency of parameters related to radiotherapy, and automatic check with use of a function of treatment planning software.

In radiotherapy, an operator checks whether the created treatment plan complies with an intended prescription and treatment policy, and whether there is no incompleteness in an image used for the treatment plan, and whether parameters fall within limiting conditions of instruments, and then uses the created treatment plan for the treatment.

These checks may be implemented by the operator, by creating a check list or the like, as described in NPL 1. Alternatively, any incompleteness of image or limitation of instruments may be checked automatically with use of a function of treatment planning software.

Any items common to the treatment plan might be automatically checked by software, by preliminarily setting presumable values. Parameters relevant to prescription or treatment policy are, however, different from patient to patient, and cannot therefore be automatized by a known method, for which operator's check is necessary.

In normal treatment planning, a single treatment plan is created for every patient. On the other hand, ART creates treatment plans multiple times, so that increase in the number of times of checking of the treatment plans would increase labor of the operator.

It is therefore an object of the present invention to provide a parameter check system, a treatment plan verification system, a treatment planning system and a radiotherapy system, as well as a program product, and a parameter check method, all capable of reducing burdens on operators and patients in radiotherapy.

### Solution to Problem

The present invention contains a plurality of means for solving the aforementioned problems. One of the means is exemplified by a parameter check system structured to compare and check parameters of a first treatment plan created with use of a first radiographic image in which a target of irradiation is captured, and parameters of a second treatment plan created with use of a second radiographic image in which the target of irradiation is captured after creation of the first treatment plan and until a date of radiation irradiation, the parameter check system including: a storage unit structured to identify and store an immutable parameter that should not change and a mutable parameter that may change, between the parameters of the first treatment plan and the parameters of the second treatment plan; and a comparison unit structured to compare, among the plurality of parameters stored in the storage unit, at least either an event regarding whether or not the immutable parameter matches between the first treatment plan and the second treatment plan, or an event regarding whether or not variation of the mutable parameter falls within a prescribed range.

### ADVANTAGEOUS EFFECTS OF INVENTION

The present invention can reduce the burdens on operators and patients in radiotherapy. Problems, structures, and effects other than those described above will be clarified by the following description of embodiments.

### BRIEF DESCRIPTION OF DRAWINGS

FIG. 1 is a drawing illustrating an outline of a structure of a radiotherapy system according to Example.

FIG. 2 is a drawing illustrating an exemplary structure of a parameter check system, in the radiotherapy system according to Example.

FIG. 3 is a drawing illustrating another exemplary structure of the parameter check system, in the radiotherapy system according to Example.

FIG. 4 is a drawing illustrating yet another exemplary structure of the parameter check system, in the radiotherapy system according to Example.

FIG. 5 is a drawing illustrating an exemplary display screen presenting results of parameter check by the parameter check system, in the radiotherapy system according to Example.

FIG. 6 is a drawing illustrating another exemplary display screen presenting results of parameter check by the parameter check system, in the radiotherapy system according to Example.

FIG. 7 is a workflow illustrating a flow of processes of a parameter check method according to Example.

### DESCRIPTION OF EMBODIMENTS

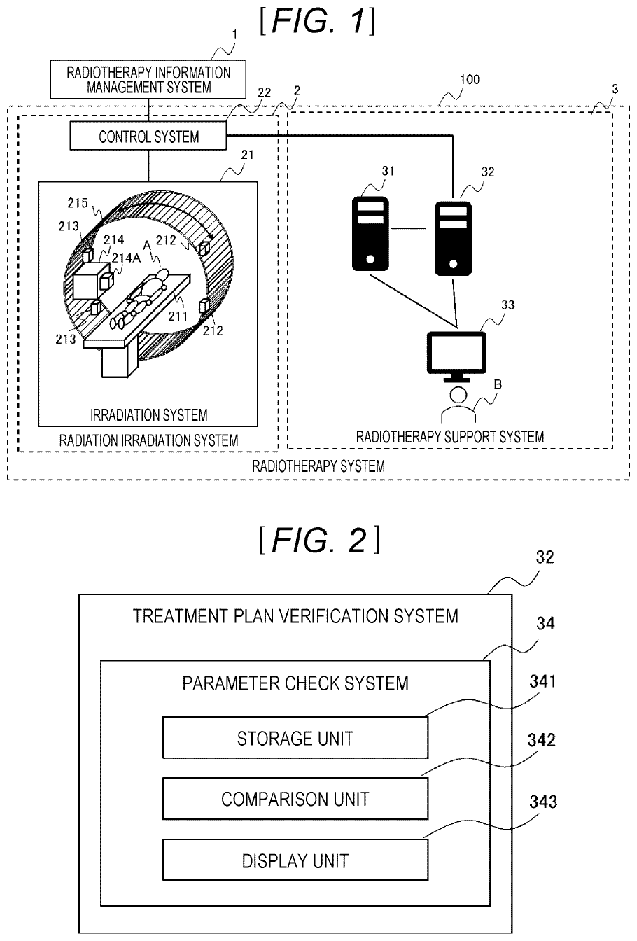

Paragraphs below will describe Examples regarding a parameter check system, a treatment plan verification system, a treatment planning system and a radiotherapy system, as well as a program product, and a parameter check method, with reference to FIGS. 1 to 7. Note in the drawings used in the present specification, the same or corresponding components are denoted by the same or similar reference numerals, so as to occasionally avoid redundant explanation for these components.

First, an overall structure of a radiotherapy system equipped with a parameter check system will be described with reference to FIG. 1. FIG. 1 illustrates an overall structure of a radiotherapy system.

A radiotherapy system 100 illustrated in FIG. 1 has a radiation irradiation system 2 and a radiotherapy support system 3. The radiotherapy system 100 is mutually connected with a radiotherapy information management system 1.

The radiotherapy information management system 1 manages treatment plan information and treatment result information received from a treatment planning system (not illustrated) operated in a hospital or the like. The radiotherapy information management system 1 sends the treatment plan information to the radiation irradiation system 2. The radiotherapy is usually performed on multiple occasions.

In this Example, the radiotherapy is separately performed on a plurality of days of treatment. The treatment plan information is a set of patient information, instrument information necessary for the treatment, dose information and the like, and typically contains information regarding radiation to be irradiated on patient A, such as an irradiation area, irradiation energy, irradiation angle, and the number of times of irradiation.

The radiation irradiation system 2 is structured to irradiate radiation, according to the treatment plan information sent from the radiotherapy information management system 1, and contains an irradiation system 21, a control system 22, and the like. The irradiation system 21 contains a couch 211 on which the patient A is supported; a radiation imaging apparatus, having a radiation generator 212 and a radiation detector 213, for photographing the patient A at the time of treatment; and a therapeutic radiation irradiation system 214 having an irradiation nozzle 214A through which the therapeutic radiation is irradiated on the patient.

The radiation irradiation system 2 of this Example photographs an internal structure of the patient A immobilized on the couch 211, with use of a radiation imaging apparatus (the radiation generator 212 and the radiation detector 213) operated by an operator B. The couch 211 is moved while referring to a photographed image, thereby moving the patient A to a planned position. The therapeutic radiation is then irradiated through the irradiation nozzle 214A on the patient A. A gantry 215 allows the irradiation nozzle 214A to circle around the patient, thereby adjusting angle of irradiation of the radiation through the irradiation nozzle 214A onto the patient A.

The radiation irradiated on the patient A is not particularly limited, for which adaptable are various types of radiation including particle beam, and electromagnetic wave such as X-ray.

Now, upon entry of a creation instruction for treatment plan by the operator B before starting the treatment, the treatment planning system 31 that has received the creation instruction creates a treatment plan that relies upon radiation irradiation, including a position of the patient A. Although having described in this Example an exemplary treatment planning by the treatment planning system 31 that constitutes the radiotherapy support system 3, some other treatment planning system installed inside or outside a hospital may alternatively receive the creation instruction to create the treatment plan.

Hereinafter, this treatment plan may be referred to as a first treatment plan. The operator B who operates the treatment planning system 31 may be a doctor responsible for radiotherapy, or may be a radiologist or the like.

The treatment planning system 31 recognizes the internal body structure of the patient A with use of an image of the patient A captured by a radiation imaging apparatus or some other imaging device in the hospital, and then creates the treatment plan including a method of irradiating therapeutic radiation. By using the created treatment plan, the patient A is laid at a planned position and irradiated with radiation by the therapeutic radiation irradiation system 214. This successfully irradiates the target patient A exactly with a planned dose, while keeping the organ around the irradiation target suppressed under planned dose.

In a flow of radiotherapy, the internal body structure of the patient would change from the date of treatment planning, over multiple days up to the last day of treatment.

A prior treatment planning, designed to use an initially created treatment plan from the first day through the last day, has preliminarily assumed such structural change, and has been planned to cause irradiation onto an area wider than the target per se, so that the target may be irradiated by a desired dose even if the structural change should occur. This has made it possible to apply a desired dose to the target, even under irradiation according to the initially created treatment plan maintained up to the last day of treatment.

On the other hand, implementation of ART, which is designed to review the internal body structure of the patient, and to accordingly modify or newly create the treatment plan, no longer needs irradiation onto such wider area allowed for the structural change, and can further reduce the dose to the organ around the irradiation target.

The ART includes offline-ART allowed for re-capture of the patient image and re-planning during intervals between a plurality of scheduled dates of treatment, and online-ART allowed for capture of the patient image and re-planning on the day of treatment while keeping the patient laid on the couch.

Although the explanation of Examples below will deal with the online-ART, this Example does not limit the treatment method, to which offline-ART or any other treatment method is applicable.

The ART prefers that the plan is quickly modified while keeping the patient A laid on the couch 211. This Example will deal with a case where a second treatment plan is created with use of an image of the patient A captured on the day of photographing with the radiation imaging apparatus, followed by re-planning with use of the radiotherapy support system 3.

The second treatment plan refers to treatment plan information re-planned by the radiotherapy support system 3, with use of a radiographic image of the patient A, acquired after the creation of the first treatment plan, and before the day of treatment or during the treatment.

The radiographic image used for creating the second treatment plan may be a radiographic image of the patient A acquired by the day on which radiotherapy is given to the patient A, or may be a radiographic image of the patient A laid on the couch 211 just on the day of treatment.

The radiotherapy support system 3 has the treatment planning system 31, a treatment plan verification system 32, a monitor 33, and the like.

The treatment planning system 31 performs re-planning with reference to an image of the patient A captured just on the day and a treatment plan created before the start of treatment, to create the second treatment plan. The system then transmits the second treatment plan to the treatment plan verification system 32. The operator B compares the second treatment plan transmitted to the treatment plan verification system 32 with the dose information in the first treatment plan created before the start of treatment, and selects a treatment plan to be used for the treatment. Concurrently with the re-planning, any preparation for the treatment same as those in the usual treatment, such as re-positioning of the couch 211 or rotation of the gantry 215, may proceed.

In the ART, the treatment policy and prescription will be categorized to the parameter which should not change (occasionally referred to as “immutable parameter”, hereinafter), since they remain unchanged between the initial planning and the re-planning. Also, a part of system setting such as the gantry angle may be categorized to the immutable parameter, since it may remain unchanged between the initial planning and the re-planning, for the purpose of saving preparation works and time for the treatment. It is, therefore, necessary to check whether the immutable parameter, out of the treatment planning parameters, matches between the initial plan and the second treatment plan.

Meanwhile, the parameter allowed for change without problem (occasionally referred to as “mutable parameter”, hereinafter) includes dose distribution ascribed to temporal changes in the irradiation target per se or an internal body path towards the irradiation target. Such changes would, however, be acceptable only within an acceptable limit, rather than being freely acceptable. It is, therefore, necessary to check whether variation of the mutable parameter, out of the treatment planning parameters, between the initial planning and the re-planning falls within an allowable range.

FIGS. 2 to 4 are drawings illustrating an exemplary structure of the parameter check system. In this Example, there is provided, as illustrated in FIG. 2, a parameter check system 34 structured to support operation for checking the treatment plan, such as parameter check in the re-planning, inside the treatment plan verification system 32 structured to verify dose distribution and the like after the re-planning.

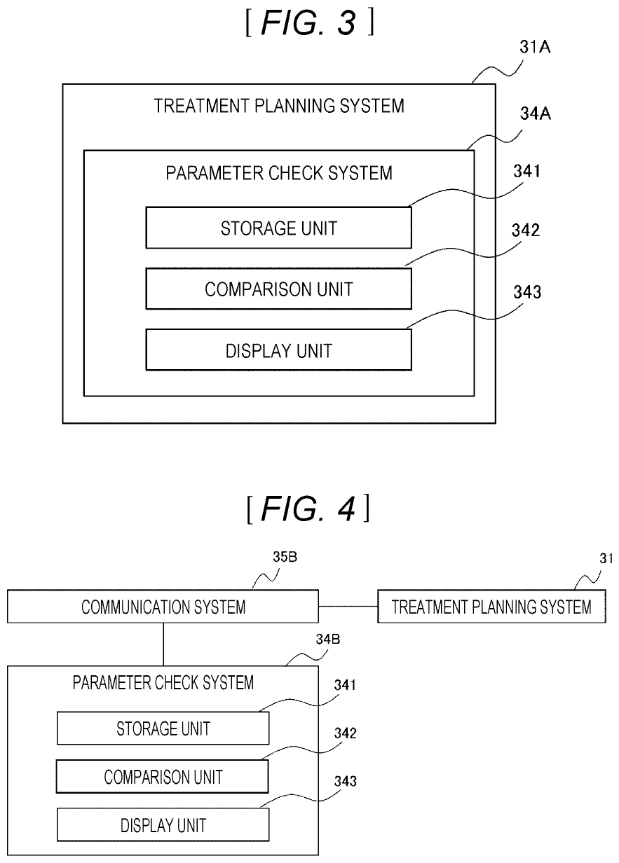

The parameter check system 34 is not always necessarily given as a structure inside the treatment plan verification system 32 as illustrated in FIG. 2, and may alternatively be given for example as illustrated in FIG. 3, in the form of a parameter check system 34A owned by a treatment planning system 31A or the treatment planning system; or, as illustrated in FIG. 4, in the form of a parameter check system 34B structured as an independent system connected via a communication system 35B to the treatment planning system 31, without special limitation.

The parameter check system 34 is structured to compare and check parameters of the first treatment plan created with use of a first radiographic image in which a target of irradiation is captured, and parameters of the second treatment plan created with use of a second radiographic image in which the target of irradiation is captured after creation of the first treatment plan and until a date of radiation irradiation, and has a storage unit 341, a comparison unit 342, and a display unit 343 as illustrated in FIG. 2. Note that the same structure is applied to the parameter check system 34A illustrated in FIG. 3 and the parameter check system 34B illustrated in FIG. 4, and will not be detailed.

The storage unit 341 identifies and stores immutable parameters that should not change, and mutable parameters that may change, between the parameters of the first treatment plan and the parameters of the second treatment plan. The storage unit 341 typically stores a treatment plan-related file for which the parameter will be checked, the parameter to be checked, a parameter check method, and a prescribed value. The treatment plan-related file to be targeted is not particularly limited as long as it is a file to be used for creating the treatment plan, and includes a treatment plan file in which radiation irradiation parameters and the like are stored, a patient image file used for the treatment planning, and an organ contour information file related to the patient image. The storage unit 341 also stores a check result given by the comparison unit 342.

The immutable parameters include at least one of patient information, prescription dose, dose constraint, treatment site, patient positioning, radiation type, irradiation technique, or preset value of irradiation apparatus including gantry angle. On the other hand, the mutable parameters include at least one of CT number, contour shape, dose distribution, spot information and control point information of therapeutic beam, or preset values of irradiation apparatus including collimator position.

Note that, among the immutable parameters, the preset values including gantry angle are classified into the mutable parameters for therapeutic instruments having unexposed gantry, such as x-ray therapeutic instrument. The immutable parameters and the mutable parameters may therefore be subject to change depending on the type of radiation or a structure of an irradiation system used for the treatment, rather than being strictly fixed.

The mutable parameters and the immutable parameters may be fixed from the time of manufacture, or may be mutable in a subsequent operation phase. In a mutable scheme, it is preferred not to accept change from the immutable parameters set so from the time of manufacture to the mutable parameters, but to accept only change from the mutable parameters to the immutable parameters.

The change may be made by an operator or a service person of a manufacturer who manufactured the system, preferably in an appropriate manner according to a treatment policy or the like of an operating entity.

The comparison unit 342 is structured to compare, among a plurality of parameters stored in the storage unit 341, at least one event whether or not the immutable parameter matches between the first treatment plan and the second treatment plan, or an event regarding whether or not variation of the mutable parameter between the first treatment plan and the second treatment plan falls within a prescribed range, according to the setting stored in the storage unit 341. The comparison unit 342 will be preferably an entity responsible for a comparison step or a comparison process structured to compare at least one event whether or not the immutable parameter matches between the first treatment plan and the second treatment plan, or an event regarding whether or not variation of the mutable parameter falls within a prescribed range.

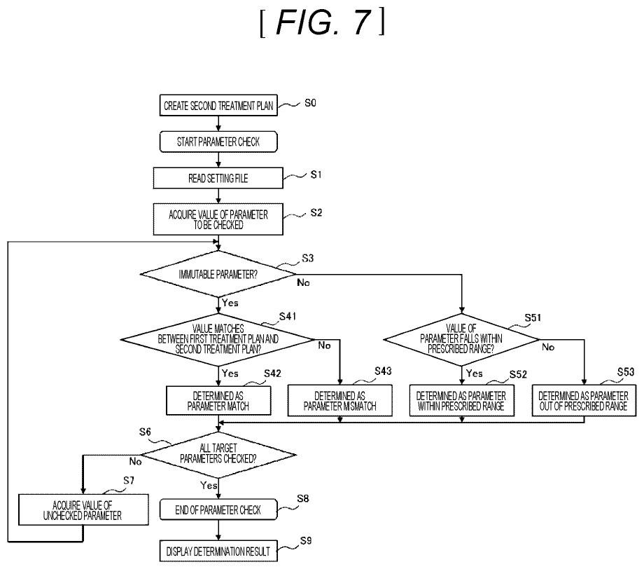

As will be detailed later with reference to FIG. 7, the parameter check method is categorized in two types, depending on whether or not the parameters are mutable between the initial treatment planning and the re-planning.

The immutable parameters are checked by comparison between the second treatment plan and the first treatment plan, meanwhile the mutable parameters are checked by whether they fall within a range of prescribed value, or by comparison between the parameters in the second treatment plan and the prescribed value. For the check by comparison with the preset value, an acceptable value is stored as the preset value. The prescribed values of the parameters may be set by numerical values or character strings, depending on types of the parameters. A plurality of prescribed values may be set, or a range of numerical value or the like may be set. Also the immutable parameters may be checked by whether or not they fall within the ranges of prescribed values.

The display unit 343 is a section responsible for display control for displaying, on a monitor 33 or the like, the results of comparison having been stored in the storage unit 341 and output from the comparison unit 342. The results may be presented while being listed in a table, or may be presented in the form of drawing, graph, or the like. When displaying information regarding the treatment plan on the monitor 33 in the treatment planning system 31 or in the treatment plan verification system 32, the information may be displayed in an appropriate display area determined in relation to the check results.

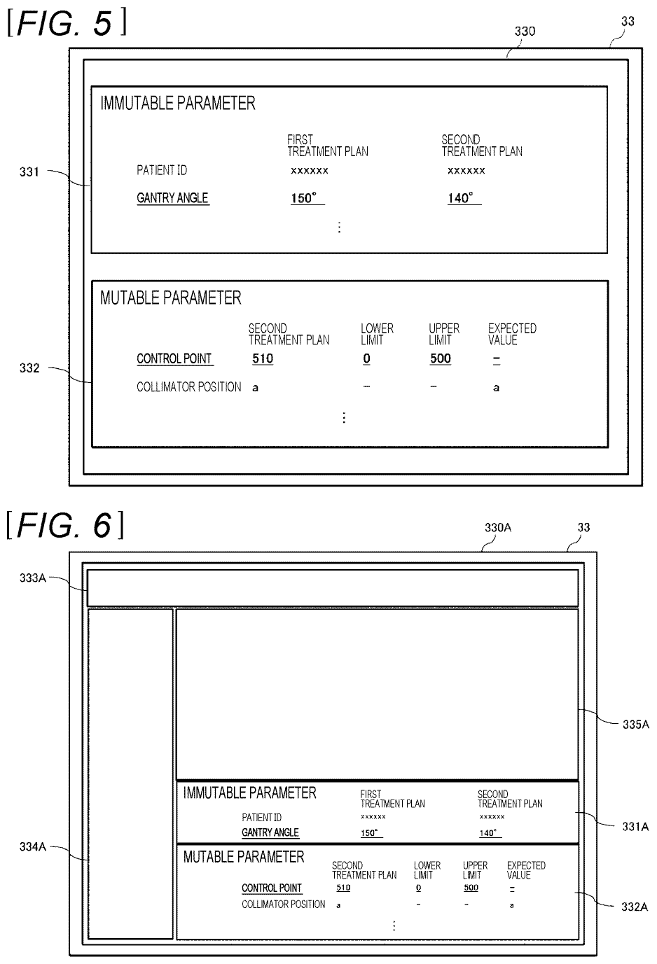

Next, an exemplary display will be described with reference to FIGS. 5 and 6. FIGS. 5 and 6 are drawings illustrating exemplary display screens presenting results of parameter check by the parameter check system.

The comparison unit 342 issues a mismatch warning to the display unit 343 when the immutable parameters do not match, and issues an out-of-prescribed-range warning to the display unit 343 if the variation of the mutable parameter falls beyond the prescribed range. Upon receiving the mismatch warning or the out-of-prescribed-range warning, the display unit 343 displays, as illustrated in FIGS. 5 and 6, the mismatch warning on a display screen 330 of the monitor 33, or displays the out-of-prescribed-range warning on the display screen 330 of the monitor 33.

On the display screen 330 that appears on the monitor 33 as illustrated in FIG. 5, displayed are an immutable parameter information display area 331 that presents “check results as to whether the immutable parameters remain unchanged”, and a mutable parameter information display area 332 that presents “check results as to whether variations of the mutable parameters fall within the prescribed range”.

In the immutable parameter information display area 331, there are, for example, parameter names such as “patient ID” and “gantry angle”, and next to them, parameter values in the first treatment plan and parameter values in the second treatment plan, which are listed side by side for the convenience of comparison. Any different value may be typically highlighted or presented in a different font color for easy recognition.

In the mutable parameter information display area 332, similarly to as in the immutable parameter information display area 331, there are, for example, parameter names such as “control point” and “collimator position”, and next to them prescribed ranges (e.g., maximum value, minimum value, or expected value), which are presented so as to enable checking whether the variation fall in the prescribed range. Similarly, any variation that falls beyond the prescribed range may be typically highlighted or presented in a different font color for easy recognition.

Display mode of the comparison results is not limited to as illustrated in FIG. 5 that presents only the comparison results. As illustrated in FIG. 6, the display unit 343 can display the mismatch warning on a display screen 330A of the monitor 33 together with other information, or can display the out-of-prescribed-range warning on the display screen 330A of the monitor 33 together with other information.

On the display screen 330A that appears on the monitor 33 as illustrated in FIG. 6, displayed are an irradiation target information display area 333A having presented therein information regarding the irradiation target, such as patient ID; a region-of-interest information display area 334A having presented therein information regarding important organ around the irradiation target; a various detailed information display area 335A having presented therein various types of information regarding the treatment plan and irradiation; an immutable parameter information display area 331A having presented therein “check results as to whether the immutable parameters remain unchanged”; and a mutable parameter information display area 332A having presented therein “check results as to whether variations of the mutable parameters fall within a prescribed range”.

In the mode of display together with other information, the immutable parameter information display area 331A and the mutable parameter information display area 332A may be displayed collectively in one display area.

The display screens 330, 330A may also present therein a warning massage, warning pop-up or the like, stating that the immutable parameter was changed, or that the variation of the mutable parameter fell beyond the prescribed range.

Also in an area other than the parameter display area, in which the immutable parameter has been changed or variation of the mutable parameter fell beyond the prescribed range, such parameters may be highlighted or presented in a different font color for easy recognition.

In the process of re-planning, the display screens 330, 330A, for presenting verification result of dose distribution and parameter verification result, may appear in such timing that the parameter verification result is displayed while calculating the dose distribution in parallel, and the verification result of dose distribution is then displayed. The timing is however not limited thereto, instead allowing both verification results are displayed at a time, or the calculation of dose distribution is started after determining that the verification results of the parameters are not problematic.

Next, an exemplary flow of the parameter check system of this Example will be explained, while referring the flow chart in FIG. 7. FIG. 7 is a workflow illustrating a flow of processes of a parameter check method.

First, upon checking the patient A has been immobilized on the couch 211, the operator B enters an instruction command for executing re-planning, to the input unit.

Having received the instruction command from the input unit, and upon acquisition of the patient image information of the day from the control system 22, the treatment planning system 31 creates the second treatment plan, with reference to the thus acquired patient image and the treatment plan (first treatment plan) having been created before the start of treatment (step S0).

Next, in the treatment plan verification system 32, upon acquisition of the second treatment plan thus created by the treatment planning system 31, the parameter check system 34 reads a setting file for parameter check having been stored in the storage unit 341 (step S1). The parameter check system 34 reads the second treatment plan, and acquires value of the parameter to be checked (step S2).

The comparison unit 342 checks whether the parameter acquired from the setting file is an immutable parameter or a mutable parameter (step S3).

If the parameter was determined to be an immutable parameter in step S3, the comparison unit 342 then checks whether the value of the parameter matches between the first treatment plan and the second treatment plan (step S41). If the value of the parameter was determined to be matched, the storage unit 341 stores the result of determination (step S42). If the value of the parameter was determined to be unmatched, the storage unit 341 stores the result of determination (step S43).

Upon determination of the mutable parameter in step S3, the comparison unit 342 then checks whether the value of the parameter falls within a range of prescribed value (step S51). If the value of the parameter was determined to fall within the range of prescribed value, the storage unit 341 stores the result of determination (step S52). If the value of the parameter was determined to fall outside the range of prescribed value, the storage unit 341 stores the result of determination (step S53). The storage unit 341 may store parameter values of the first treatment plan and the second treatment plan, in addition to the determination result.

The comparison unit 342 of the parameter check system 34 determines whether or not all the target parameters have been checked (step S6). If any unchecked parameter was determined to remain, the comparison unit 342 acquires a value of the next unchecked parameter (step S7). If all the target parameters were determined to be checked, the parameter check is ended (step S8).

The display unit 343 of the parameter check system 34 displays the results of determination of the target parameters on the monitor 33 (step S9).

Next, effects of this Example will be described.

The aforementioned parameter check system 34 of this Example is structured to compare and check parameters of a first treatment plan created with use of a first radiographic image in which a target of irradiation is captured, and parameters of a second treatment plan created with use of a second radiographic image in which the target of irradiation is captured after creation of the first treatment plan and until a date of radiation irradiation, the parameter check system 34 includes: a storage unit 341 structured to identify and store an immutable parameter that should not change and a mutable parameter that may change, between the parameters of the first treatment plan and the parameters of the second treatment plan; and a comparison unit 342 structured to compare, among the plurality of parameters stored in the storage unit 341, at least either an event regarding whether or not the immutable parameter matches between the first treatment plan and the second treatment plan, or an event regarding whether or not variation of the mutable parameter falls within a prescribed range.

In radiotherapy, a created treatment plan needs to be checked for the soundness and validity, such as whether the created treatment plan is in accordance with the treatment policy, after the planning and before the irradiation on a patient. The present structure can automatize checking of the soundness and validity of the plan, and can reduce the burdens on operators and patients.

Widely-known radiotherapy, in which irradiation does not take place immediately after the treatment planning, can use a plenty of time for checking the treatment plan. Online-ART, however, needs rapid check of the treatment plan, since the treatment plan is created while keeping the patient laid on the couch. If the check delays, the treatment time will increase, and the burden on the patient will increase due to an effort for maintaining the posture during the treatment. The present structure can shorten the treatment time, and can reduce the burden on the patient.

The ART is structured to perform the re-planning while keeping the target site of treatment or treatment purpose unchanged. Moreover, the online-ART can save the time required for setup or instrument setting for the second time, by the re-planning while keeping the parameters relevant to the setup or instrument setting unchanged from those in the initial planning. This, however, needs checking whether the parameters remain unchanged from those in the initial treatment planning, in addition to the usual checking at the time of radiotherapy. Although the prior technique was hard to check consistency between the aforementioned treatment plans, the present structure enables checking of the consistency, and can implement the online-ART without increasing the labor of the operator due to setup or instrument setting.

Since the comparison unit 342 is structured to output the comparison results of the immutable parameters and the mutable parameters, and since the parameter check system further includes the display unit 343 structured to allow the monitor 33 to display the comparison results output from the comparison unit 342, so that the operator can check how the comparison results turn out, whereby the labor will be reduced.

The comparison unit 342 also issues the mismatch warning to the display unit 343 when the immutable parameters do not match, and issues the out-of-prescribed-range warning to the display unit 343 if the variation of the mutable parameter falls beyond the prescribed range. This makes the operator more easily recognize the comparison results.

The display unit 343 displays the mismatch warning together with other information on the display screen 330A of the monitor 33, and displays the out-of-prescribed-range warning together with other information on the display screen 330A of the monitor 33, thereby allowing the system to appropriately select a display unit same as the treatment plan information associated with the parameter, and to output the parameter check result. This enables the operator to determine validity of the result of treatment planning, and to check soundness of the parameters at the same time, so that the treatment time is further shortened, thus making the treatment less time-consuming, and less labor-consuming for the operator.

### Others

Note that the present invention is not limited to the above embodiments, and is allowed for various modifications and applications. The aforementioned Example has been detailed merely for easy understanding of the present disclosure, and is not always necessarily limited to those having all structures described above.

Example of the present invention may have the following aspects.

(1) A parameter check system structured to compare and check parameters of a first treatment plan created with use of a first radiographic image in which a target of irradiation is captured, and parameters of a second treatment plan created with use of a second radiographic image in which the target of irradiation is captured after creation of the first treatment plan and until a date of radiation irradiation, the parameter check system including: a storage unit structured to identify and store an immutable parameter that should not change and a mutable parameter that may change, between the parameters of the first treatment plan and the parameters of the second treatment plan; and a comparison unit structured to compare, among the plurality of parameters stored in the storage unit, at least either an event regarding whether or not the immutable parameter matches between the first treatment plan and the second treatment plan, or an event regarding whether or not variation of the mutable parameter falls within a prescribed range.

(2) The parameter check system according to (1), wherein the comparison unit is structured to output a comparison result of the immutable parameter and the mutable parameter, and the parameter check system further includes a display unit structured to allow a display device to display the comparison result output from the comparison unit.

(3) The parameter check system according to (1) or (2), wherein the comparison unit issues a mismatch warning to the display unit, if the immutable parameter does not match.

(4) The parameter check system according to (3), wherein the display unit is structured to allow the display device to display, on its display screen, the mismatch warning together with other information.

(5) The parameter check system according to any one of (2) to (4), wherein the comparison unit is structured to issue an out-of-prescribed-range warning to the display unit, if the variation of the mutable parameter falls beyond the prescribed range.

(6) The parameter check system according to (5), wherein the display unit is structured to allow the display device to display, on its display screen, the out-of-prescribed-range warning together with other information.

(7) The parameter check system according to any one of (1) to (6), wherein the immutable parameter includes at least one of patient information, prescription dose, dose constraint, treatment site, patient positioning, radiation type, irradiation technique, or preset values of irradiation apparatus including gantry angle.

(8) The parameter check system according to any one of (1) to (7), wherein the mutable parameter includes at least one of CT number, contour shape, dose distribution, spot information and control point information of therapeutic beam, or preset values of irradiation apparatus including collimator position.

### REFERENCE SIGNS LIST

1 radiotherapy information management system
2 radiation irradiation system
3 radiotherapy support system
21 irradiation system
22 control system
31, 31A treatment planning system
32 treatment plan verification system
33 monitor
34, 34A, 34B parameter check system
35B communication system
100 radiotherapy system
211 couch
212 radiation generator
213 radiation detector
214 therapeutic radiation irradiation system
214A irradiation nozzle
215 gantry
330, 330A display screen
331, 331A immutable parameter information display area
332, 332A mutable parameter information display area
333A irradiation target information display area
334A region-of-interest information display area
335A detailed information display area
341 storage unit
342 comparison unit
343 display unit

1. A parameter check system structured to compare and check parameters of a first treatment plan created with use of a first radiographic image in which a target of irradiation is captured, and parameters of a second treatment plan created with use of a second radiographic image in which the target of irradiation is captured after creation of the first treatment plan and until a date of radiation irradiation, the parameter check system comprising:
a storage unit structured to identify and store an immutable parameter that should not change and a mutable parameter that may change, between the parameters of the first treatment plan and the parameters of the second treatment plan; and
a comparison unit structured to compare, among the plurality of parameters stored in the storage unit, at least either an event regarding whether or not the immutable parameter matches between the first treatment plan and the second treatment plan, or an event regarding whether or not variation of the mutable parameter falls within a prescribed range.

2. The parameter check system according to claim 1, wherein
the comparison unit is structured to output a comparison result of the immutable parameter and the mutable parameter, and
the parameter check system further comprises a display unit structured to allow a display device to display the comparison result output from the comparison unit.

3. The parameter check system according to claim 2, wherein
the comparison unit issues a mismatch warning to the display unit, if the immutable parameter does not match.

4. The parameter check system according to claim 3, wherein
the display unit is structured to allow the display device to display, on its display screen, the mismatch warning together with other information.

5. The parameter check system according to claim 2, wherein
the comparison unit is structured to issue an out-of-prescribed-range warning to the display unit, if the variation of the mutable parameter falls beyond the prescribed range.

6. The parameter check system according to claim 5, wherein
the display unit is structured to allow the display device to display, on its display screen, the out-of-prescribed-range warning together with other information.

7. The parameter check system according to claim 1, wherein
the immutable parameters include at least one of patient information, prescription dose, dose constraint, treatment site, patient positioning, radiation type, irradiation technique, or preset values of irradiation apparatus including gantry angle.

8. The parameter check system according to claim 1, wherein
the mutable parameters include at least one of CT number, contour shape, dose distribution, spot information and control point information of therapeutic beam, or preset values of irradiation apparatus including collimator position.

9. A treatment plan verification system comprising the parameter check system according to claim 1.

10. A treatment planning system comprising the parameter check system according to claim 1.

11. A radiotherapy system comprising the parameter check system according to claim 1.

12. A program product for comparing and checking parameters of a first treatment plan created with use of a first radiographic image in which a target of irradiation is captured, and parameters of a second treatment plan created with use of a second radiographic image in which the target of irradiation is captured after creation of the first treatment plan and until a date of radiation irradiation, the program product being structured to allow a parameter check system to execute:
an identification step for identifying an immutable parameter that should not change and a mutable parameter that may change, between the parameters of the first treatment plan and the parameters of the second treatment plan; and
a comparison step for comparing at least either an event regarding whether or not the immutable parameter matches between the first treatment plan and the second treatment plan, or an event regarding whether or not variation of the mutable parameter falls within a prescribed range.

13. A parameter check method for comparing and checking parameters of a first treatment plan created with use of a first radiographic image in which a target of irradiation is captured, and parameters of a second treatment plan created with use of a second radiographic image in which the target of irradiation is captured after creation of the first treatment plan and until a date of radiation irradiation, the method comprising:
a comparison step for comparing at least either an event regarding whether or not the immutable parameter, which should not change between the parameter of the first treatment plan and the parameter of the second treatment plan, matches between the first treatment plan and the second treatment plan, or an event regarding whether the mutable parameter, which may change between the parameters of the first treatment plan and the parameters of the second treatment plan, falls within a prescribed range.
